(19) United States
(12) Patent Application Publication
(10) Pub. No.: US 2017/0303775 A1
(43) Pub. Date: Oct. 26, 2017

(54) ENDOSCOPE APPARATUS AND ENDOSCOPE SYSTEM

(71) Applicant: OLYMPUS CORPORATION, Tokyo (JP)

(72) Inventors: Hiroki UCHIYAMA, Tokyo (JP); Toshiaki WATANABE, Tokyo (JP); Misa TSURUTA, Tokyo (JP); Yuichi TAKEUCHI, Tokyo (JP)

(73) Assignee: OLYMPUS CORPORATION, Tokyo (JP)

(21) Appl. No.: 15/646,498

(22) Filed: Jul. 11, 2017

Related U.S. Application Data
(63) Continuation of application No. PCT/JP2016/061234, filed on Apr. 6, 2016.

(30) Foreign Application Priority Data
Sep. 18, 2015 (JP) 2015-185196

(51) Int. Cl. A61B 1/04; A61B 1/06; A61B 1/05; A61B 1/00; A61B 1/045; A61B 1/07

(52) U.S. Cl. CPC A61B 1/043; A61B 1/07; A61B 1/0646; A61B 1/0638; A61B 1/00006; A61B 1/045; A61B 1/00045; A61B 1/053

(57) ABSTRACT

An endoscope apparatus enables observation using reflected light of white light and observation using fluorescence, and includes: an insertion portion configured to be insertable into a subject; an objective optical system; a transmitting optical system; a camera unit; an optical splitter configured to split light exiting through the transmitting optical system; an optical filter disposed in the transmitting optical system or the camera unit, the optical filter being configured to transmit the reflected light of the white light and the fluorescence and block the reflected light of the excitation light; a first image pickup device for picking up an image of the reflected light of the white light exiting through the optical filter and the optical splitter; and a second image pickup device for picking up an image of the fluorescence exiting through the optical filter and the optical splitter.

### CROSS REFERENCE TO RELATED APPLICATION

This application is a continuation application of PCT/JP2016/061234 filed on Apr. 6, 2016 and claims benefit of Japanese Application No. 2015-185196 filed in Japan on Sep. 18, 2015, the entire contents of which are incorporated herein by this reference.

### BACKGROUND OF THE INVENTION

### 1. Field of the Invention

The present invention relates to an endoscope apparatus and an endoscope system, and specifically relates to an endoscope apparatus and an endoscope system that are used for fluorescence observation.

### 2. Description of the Related Art

In endoscopic observations in a medical field, for example, white light observation, which is an observation technique of observing a state of an object such as living tissue present in a subject based on reflected light emitted when white light is radiated to the object, has conventionally been performed.

Also, in endoscopic observations in the medical field, for example, fluorescence observation, which is an observation technique of making diagnosis of, e.g. whether or not a lesion site is included in a desired observed site based on a state of generation of fluorescence when excitation light for exciting either a fluorescent dye administered into a subject or a predetermined fluorescent substance present in cells of the subject is radiated to the desired site to be observed, has conventionally been performed.

Then, for example, EP Patent Application Publication No. 2122331 discloses a configuration that can be used for both the white light observation and the fluorescence observation mentioned above.

More specifically, EP Patent Application Publication No. 2122331 discloses a system that can perform image pickup of reflected light of white light to provide a reflected light image and image pickup of fluorescence emitted from plural types of fluorescent dyes excited by excitation light in a plurality of mutually-different wavelength bands to provide a fluorescence image.

### SUMMARY OF THE INVENTION

An endoscope apparatus according to an aspect of the present invention is an endoscope apparatus enabling observation using reflected light of white light, the reflected light being generated in response to radiation of the white light to an object present in a subject, observation using first fluorescence generated in response to radiation of first excitation light to a first fluorescent dye administered into the subject, and observation using second fluorescence generated in response to radiation of second excitation light to a second fluorescent dye administered into the subject, the endoscope apparatus including: an insertion portion configured to be insertable into the subject; an objective optical system provided in a distal end portion of the insertion portion, and configured to allow entrance of the reflected light of the white light, the first fluorescence, the second fluorescence, reflected light of the first excitation light, the reflected light being generated in response to the radiation of the first excitation light, and reflected light of the second excitation light, the reflected light being generated in response to the radiation of the second excitation light, as return light; a transmitting optical system provided on a proximal end side relative to the objective optical system in the insertion portion, the transmitting optical system including a plurality of lenses, and being configured to transmit the return light obtained from the objective optical system; a camera unit configured to be detachably attachable to the insertion portion, the camera unit including a plurality of image pickup devices configured to allow entrance of the return light travelling through the transmitting optical system; an optical splitter provided in the camera unit and configured to split the return light exiting through the transmitting optical system into light in a wavelength band of the white light and light in a wavelength band that is different from the wavelength band of the white light and cause an exit of the light in the wavelength band of the white light and an exit of the light in the wavelength band that is different from the wavelength band of the white light; an optical filter disposed between the plurality of lenses in the transmitting optical system or disposed at a predetermined position on the return light entrance side of the optical splitter in the camera unit, the optical filter being formed to have an optical characteristic of transmitting the reflected light of the white light, the first fluorescence and the second fluorescence and blocking the reflected light of the first excitation light and the reflected light of the second excitation light; a first image pickup device for picking up an image of the reflected light of the white light exiting through the optical filter and the optical splitter, the first image pickup device being included in the plurality of image pickup devices; and a second image pickup device for picking up an image of the first fluorescence and the second fluorescence exiting through the optical filter and the optical splitter, the second image pickup device being included in the plurality of image pickup devices.

An endoscope system according to an aspect of the present invention includes: a light source apparatus configured to be capable of supplying white light for illuminating an object present in a subject, first excitation light for exciting a first fluorescent dye administered into the subject to generate first fluorescence, and second excitation light for exciting a second fluorescent dye administered into the subject to generate second fluorescence; an insertion portion configured to be insertable into the subject; an objective optical system provided in a distal end portion of the insertion portion, and configured to allow entrance of reflected light of the white light generated in response to radiation of the white light to the object, the first fluorescence, the second fluorescence, reflected light of the first excitation light, the reflected light being generated in response to the radiation of the first excitation light, and reflected light of the second excitation light, the reflected light being generated in response to the radiation of the second excitation light, as return light; a transmitting optical system provided on a proximal end side relative to the objective optical system in the insertion portion, the transmitting optical system including a plurality of lenses, and being configured to transmit the return light obtained from the objective optical system; a camera unit configured to be detachably attachable to the insertion portion, the camera unit including a plurality of image pickup devices configured to allow entrance of the return light travelling through the transmitting optical system; an optical splitter provided in the camera unit and configured to split the return light exiting through the transmitting optical system into light in a wavelength band of the white light and light in a wavelength band that is different from the wavelength band of the white light and cause an exit of the light in the wavelength band of the white light and an exit of the light in the wavelength band that is different from the wavelength band of the white light; an optical filter disposed between the plurality of lenses in the transmitting optical system or disposed at a predetermined position on the return light entrance side of the optical splitter in the camera unit, the optical filter being formed to have an optical characteristic of transmitting the reflected light of the white light, the first fluorescence and the second fluorescence and blocking the reflected light of the first excitation light and the reflected light of the second excitation light; a first image pickup device for picking up an image of the reflected light of the white light exiting through the optical filter and the optical splitter, the first image pickup device being included in the plurality of image pickup devices; and a second image pickup device for picking up an image of the first fluorescence and the second fluorescence exiting through the optical filter and the optical splitter, the second image pickup device being included in the plurality of image pickup devices.

### BRIEF DESCRIPTION OF THE DRAWINGS

FIG. 1 is a diagram illustrating a configuration of a major part of an endoscope system according to a first embodiment;

FIG. 2 is a diagram for describing an example of a specific configuration of the endoscope system according to the first embodiment;

FIG. 3 is a diagram indicating an example of an optical characteristic of an optical filter provided in an endoscope apparatus according to the first embodiment;

FIG. 4 is a diagram indicating an example of wavelength bands of light emitted from respective light sources provided in a light source apparatus according to the first embodiment;

FIG. 5 is a diagram illustrating a configuration of a major part of an endoscope system according to a modification of the first embodiment;

FIG. 6 is a diagram illustrating an example of the configuration of the light source apparatus according to the first embodiment, the example being different from the example in FIG. 2;

FIG. 7 is a diagram illustrating an example of a configuration of a rotary filter provided in the light source apparatus in FIG. 6;

FIG. 8 is a diagram indicating an example of a transmission characteristic of a white light observation filter provided in the rotary filter in FIG. 7;

FIG. 9 is a diagram indicating an example of a transmission characteristic of a fluorescence observation filter provided in the rotary filter in FIG. 7; and

FIG. 10 is a diagram illustrating a configuration of a major part of an endoscope system according to a second embodiment.

### DETAILED DESCRIPTION OF THE PREFERRED EMBODIMENTS

Embodiments of the present invention will be described below with reference to the drawings.

### First Embodiment

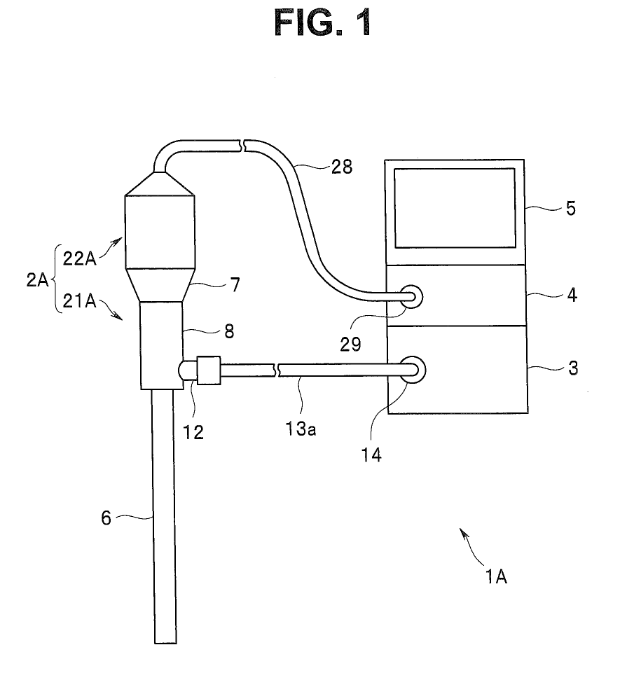

FIGS. 1 to 9 relate to a first embodiment and a modification of the first embodiment of the present invention.

As illustrated in FIG. 1, an endoscope system 1A includes an endoscope apparatus 2A configured to be inserted into a subject and output an image obtained by image pickup of an object such as living tissue in the subject, a light source apparatus 3 configured to supply light to be radiated to the object, to the endoscope apparatus 2A, a video processor 4 configured to perform predetermined image processing on the image outputted from the endoscope apparatus 2A to generate, e.g., an observation image and output the observation image or the like, and a display apparatus 5 configured to display the observation image or the like outputted from the video processor 4, on a screen. FIG. 1 is a diagram illustrating a configuration of a major part of an endoscope system according to a first embodiment.

The endoscope apparatus 2A is configured to enable observation using reflected light of white light, the reflected light being generated in response to radiation of the white light, to an object present in a subject, and observation using fluorescence generated in response to radiation of excitation light to a fluorescent dye administered into the subject. Also, the endoscope apparatus 2A is configured to include an optical viewing tube 21A including an elongated insertion portion 6, and a camera unit 22A that is detachably attachable to an eyepiece portion 7 of the optical viewing tube 21A.

The optical viewing tube 21A is configured to include the elongated insertion portion 6 that is insertable into a subject, a grasping portion 8 provided at a proximal end portion of the insertion portion 6, and the eyepiece portion 7 provided at a proximal end portion of the grasping portion 8.

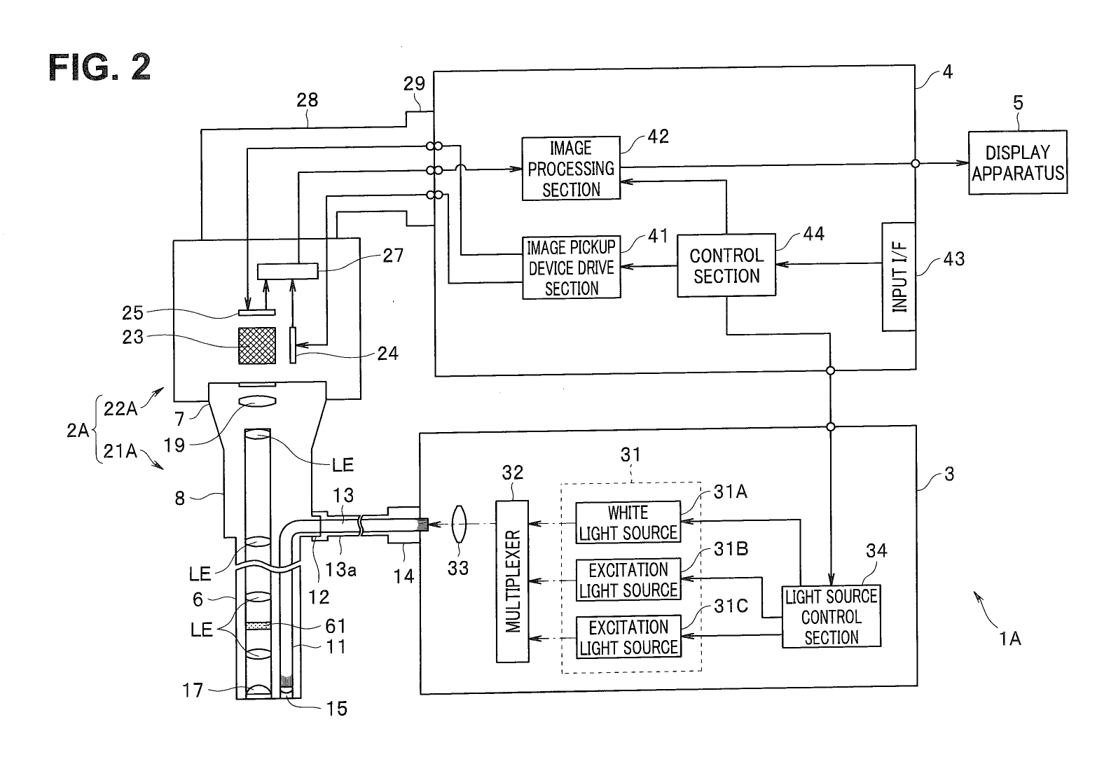

As illustrated in FIG. 2, a light guide 11 for transmitting light supplied via a cable 13a is inserted inside the insertion portion 6. FIG. 2 is a diagram for describing an example of a specific configuration of the endoscope system according to the first embodiment.

As illustrated in FIG. 2, an exit end portion of the light guide 11 is disposed in the vicinity of an illumination lens 15 in a distal end portion of the insertion portion 6. Also, an entrance end portion of the light guide 11 is disposed in a light guide pipe sleeve 12 provided in the grasping portion 8.

As illustrated in FIG. 2, a light guide 13 for transmitting light supplied from the light source apparatus 3 is inserted inside the cable 13a. Also, at one end portion of the cable 13 a, a connection member (not illustrated) that is detachably attachable to the light guide pipe sleeve 12 is provided. Also, at the other end portion of the cable 13a, a light guide connector 14 that is detachably attachable to the light source apparatus 3 is provided.

In the distal end portion of the insertion portion 6, an illumination lens 15 for causing the light transmitted by the light guide 11 to exit to the outside, and an objective lens 17 for obtaining an optical image according to the light entered from the outside are provided. Also, in a distal end face of the insertion portion 6, an illumination window (not illustrated) in which the illumination lens 15 is disposed and an objective window (not illustrated) in which the objective lens 17 is disposed are provided adjacent to each other.

As illustrated in FIG. 2, a relay lens 18 including a plurality of lenses LE for transmitting an optical image obtained by the objective lens 17 to the eyepiece portion 7 is provided inside the insertion portion 6. In other words, the relay lens 18 is configured to have a function as a transmitting optical system configured to transmit light entered from the objective lens 17.

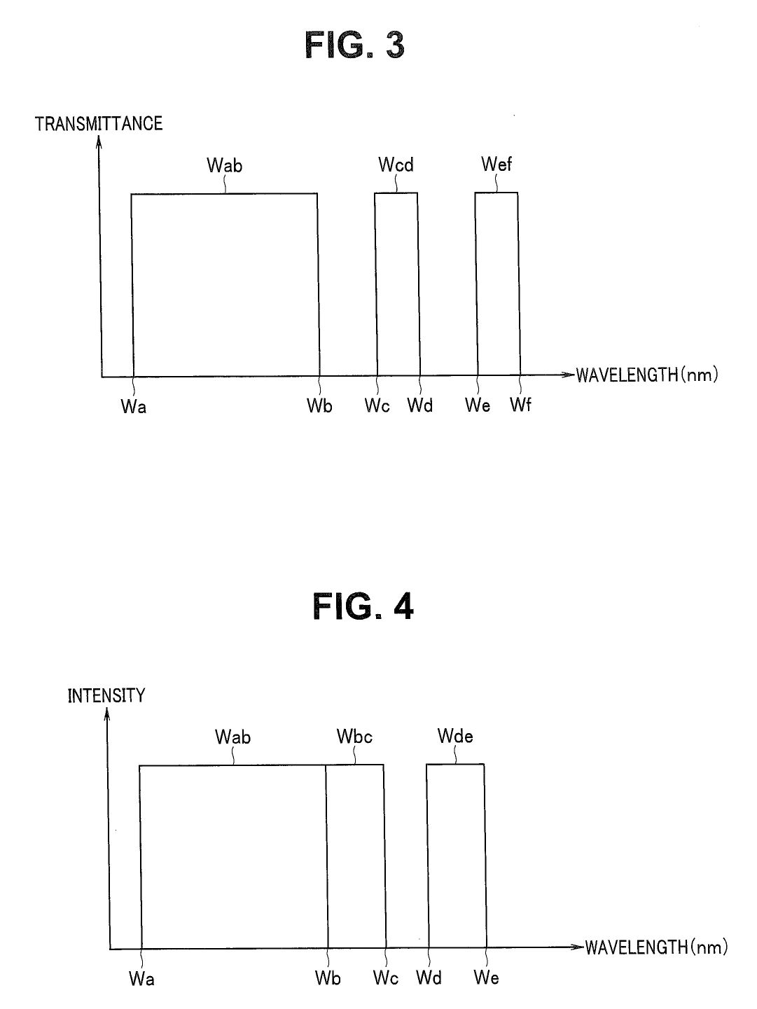

Also, an optical filter 61 formed to have an optical characteristic such as indicated in FIG. 3 is provided between predetermined two lenses LE from among the plurality of lenses LE included in the relay lens 18. In other words, the optical filter 61 in the present embodiment is disposed on an optical path on which light entered from the objective optical system 17 travels to a later-described optical splitter 23 through the relay lens 18. FIG. 3 is a diagram indicating an example of an optical characteristic of an optical filter provided in an endoscope apparatus according to the first embodiment.

More specifically, for example, as illustrated in FIG. 3, the optical filter 61 is formed to have an optical characteristic of transmitting light included in any of three wavelength bands that are a wavelength band Wab corresponding to a band of from a wavelength Wa belonging to a blue range to a wavelength Wb belonging to a red range, a wavelength band Wcd corresponding to a band of from a wavelength Wc belonging to a near-infrared range to a wavelength Wd that is longer than the wavelength Wc, and a wavelength band Wef corresponding to a band of from a wavelength We that is longer than the wavelength Wd to a wavelength Wf that is longer than the wavelength We and blocking light included in a wavelength band other than the three wavelength bands.

As illustrated in FIG. 2, an eyepiece lens 19 for enabling an optical image transmitted by the relay lens 18 to be observed with a naked eye is provided inside the eyepiece portion 7.

The camera unit 22A is configured to include the optical splitter 23, image pickup devices 24 and 25, and a signal processing circuit 27.

The optical splitter 23 is configured to include one or more optical members, for example, dichroic mirrors or prisms, and split light exiting through the eyepiece lens 19 into light in a plurality of mutually-different wavelength bands and cause respective exits of the light to the image pickup devices 24 and 25. More specifically, the optical splitter 23 is configured to split light exiting through the eyepiece lens 19 into light in a first wavelength band, which is light in a band of wavelengths that are equal to or shorter than the wavelength Wb, and light in a second wavelength band, which is light in a band of wavelengths that are longer than the wavelength Wb, and cause an exit of the light in the first wavelength band to the image pickup device 24 and cause an exit of the light in the second wavelength band to the image pickup device 25.

The image pickup device 24 is configured by, for example, a color CCD with a primary color or complimentary color filter provided on an image pickup surface. Also, the image pickup device 24 is configured to perform image pickup operation according to an image pickup device drive signal outputted from the video processor 4. Also, the image pickup device 24 is configured to perform image pickup of light exiting through the optical splitter 23, the light being in a band of wavelengths that are equal to or shorter than the wavelength Wb, and produce and output an image according to the light subjected to the image pickup.

The image pickup device 25 is configured by, for example, a highly-sensitive monochrome CCD. Also, the image pickup device 25 is configured to perform image pickup operation according to an image pickup device drive signal outputted from the video processor 4. Also, the image pickup device 25 is configured to perform image pickup of light exiting through the optical splitter 23, the light being in a band of wavelengths that are longer than the wavelength Wb, and produce and output an image according the light subjected to the image pickup.

The signal processing circuit 27 is configured to perform predetermined signal processing such as, for example, correlated double sampling processing, gain adjustment processing and A/D conversion processing on the respective images outputted from the image pickup devices 24 and 25. Also, the signal processing circuit 27 is configured to output the image resulting from the aforementioned predetermined signal processing to the video processor 4 to which a signal cable 28 is connected.

The light source apparatus 3 is configured to include a light emission section 31, a multiplexer 32, a condenser lens 33 and a light source control section 34. Also, the light emission section 31 is configured to include a white light source 31A, and excitation light sources 31B and 31C.

The white light source 31A is configured to include, for example, a lamp or an LED, and emit WL light, which is white light in the wavelength band Wab (see FIG. 4). Also, the white light source 31A is configured to switch into an on state or an off state according to control performed by the light source control section 34. Also, the white light source 31A is configured to generate WL light, an intensity or an amount of which is subject to control performed by the light source control section 34, in the on state. FIG. 4 is a diagram indicating an example of wavelength bands of light emitted from respective light sources provided in a light source apparatus according to the first embodiment.

The excitation light source 31B is configured to include, for example, a lamp or an LED and emit EA light, which is excitation light in a wavelength band Wbc corresponding to a band of from the wavelength Wb to the wavelength We (see FIG. 4). Also, the excitation light source 31B is configured to switch into an on state or an off state according to control performed by the light source control section 34. Also, the excitation light source 31B is configured to generate EA light, an intensity or an amount of which is subject to control performed by the light source control section 34, in the on state.

The excitation light source 31C is configured to include, for example, a lamp or an LED and emit EB light, which is excitation light in a wavelength band Wde corresponding to a band of from the light wavelength Wd to the wavelength We (see FIG. 4). Also, the excitation light source 31C is configured to switch into an on state or an off state according to control performed by the light source control section 34. Also, the excitation light source 31C is configured to generate EB light, an intensity or an amount of which is subject to control performed by the light source control section 34, in the on state.

Here, it is only necessary that the optical filter 61 and the light emission section 31 in the present embodiment be configured to prevent the wavelength band Wbc or Wde and the wavelength band Wcd or Wef from overlapping each other and make the wavelength bands Wcd and Wef different from each other. Therefore, the optical filter 61 and the light emission section 31 in the present embodiment may be, for example, configured to make the wavelength bands Wab and Wbc partially overlap or configured to make the wavelength bands Wbc and Wde partially overlap.

The multiplexer 32 is configured to be capable of multiplexing respective light emitted from the light emission section 31 and causing the resulting light to enter the condenser lens 33.

The condenser lens 33 is configured to collect light entered through the multiplexer 32 and output the light to the light guide 13.

The light source control section 34 is configured to perform control of the respective light sources in the light emission section 31 based on an illumination control signal outputted from the video processor 4.

In other words, the above-described configuration of the light source apparatus 3 enables supply of WL light, which is white light for illuminating an object present in a subject, and EA light and EB light, each of which is excitation light for exciting a fluorescent dye administered into the subject to generate fluorescence, to the optical viewing tube 21A in the endoscope apparatus 2A.

The video processor 4 is configured to include an image pickup device drive section 41, an image processing section 42, an input IN (interface) 43 and a control section 44.

The image pickup device drive section 41 is configured to include, for example, a driver circuit. Also, the image pickup device drive section 41 is configured to produce an image pickup device drive signal according to control performed by the control section 44 and output the image pickup device drive signal.

The image processing section 42 is configured to include, for example, an image processing circuit. Also, the image processing section 42 is configured to, according to control performed by the control section 44, subject an image outputted from the endoscope apparatus 2A when a white light observation mode is set, to predetermined image processing such as tone correction processing to produce a white light observation image and output the produced white light observation image to the display apparatus 5. Also, the image processing section 42 is configured to, according to control performed by the control section 44, subject an image outputted from the endoscope apparatus 2A when a fluorescence observation mode is set, to predetermined image processing such as tone correction processing to produce a fluorescence observation image and output the produced fluorescence observation image to the display apparatus 5.

The input I/F 43 is configured to include one or more switches and/or buttons that each enable provision of, e.g., an instruction according to operation performed by a user. More specifically, the input I/F 43 is configured to include, for example, an observation mode changeover switch (not illustrated) that enables provision of an instruction for setting (switching) an observation mode of the endoscope system 1A to either the white light observation mode or the fluorescence observation mode according to operation performed by a user.

The control section 44 is configured to include, for example, a control circuit such as a CPU or an FPGA (field programmable gate array). Also, the control section 44 is configured to produce an illumination control signal for causing an exit of light according to the observation mode of the endoscope system 1A and output the illumination control signal to the light source control section 34 based on an instruction provided via the observation mode changeover switch of the input I/F 43. Also, the control section 44 is configured to control the image pickup device drive section 41 and the image processing section 42 to perform operation according to the observation mode of the endoscope system 1A, based on an instruction provided via the observation mode changeover switch of the input I/F 43.

The display apparatus 5 is configured to include, for example, an LCD (liquid-crystal display) and be capable of displaying, e.g., an observation image outputted from the video processor 4.

Next, operation, etc., of the endoscope system 1A according to the present embodiment will be described. Here, the present embodiment will be described taking the case where a fluorescent dye FLA having a fluorescence characteristic of emitting FA light, which is fluorescence in the wavelength band Wcd (near-infrared range), in response to radiation of EA light and a fluorescent dye FLB having a fluorescence characteristic of emitting FB light, which is fluorescence in the wavelength band Wef (near-infrared range), in response to radiation of EB light are administered into a subject at mutually-different timings, as an example.

First, a user such as a surgeon connects the respective sections of the endoscope system 1A and turns on the power, and then operates the input I/F 43 to provide an instruction to set the observation mode of the endoscope system 1A to the white light observation mode.

Upon detection of the setting to the white light observation mode, the control section 44 produces an illumination control signal for causing an exit of WL light from the light source apparatus 3 and outputs the illumination control signal to the light source control section 34. Also, upon detection of the setting to the white light observation mode, the control section 44 controls the image pickup device drive section 41 to drive the image pickup device 24 in the camera unit 22A and stop driving of the image pickup device 25 in the camera unit 22A.

In response to the illumination control signal outputted from the control section 44, the light source control section 34 performs control for bringing the white light source 31A into the on state and also performs control for bringing the excitation light source 31B and the excitation light source 31C into the off state. Also, according to control performed by the control section 44, the image pickup device drive section 41 produces an image pickup device drive signal for causing image pickup operation and outputs the image pickup device drive signal to the image pickup device 24, and produces an image pickup device drive signal for stopping image pickup operation and outputs the image pickup device drive signal to the image pickup device 25.

Then, as a result of the above-described operation being performed in the light source control section 34, WL light is radiated to an object, and WLR light, which is reflected light emitted from the object in response to the radiation of the WL light, enters from the objective lens 17 as return light, and the WLR light exiting through the objective lens 17 enters the relay lens 18.

Here, according to the above-described configuration of the optical viewing tube 21A, the optical filter 61 provided in the relay lens 18 has an optical characteristic such as indicated in FIG. 3 as an example, and thus, the WLR light in the wavelength band Wab enters the optical splitter 23 through the eyepiece lens 19 side.

Therefore, when the observation mode of the endoscope system 1A is set to the white light observation mode, the WLR light exiting through the optical splitter 23 is subjected to image pickup by the image pickup device 24, and a reflected light image RI, which is an image obtained as a result of the image pickup of the WLR light, is outputted to the image processing section 42 through the signal processing circuit 27.

According to control performed by the control section 44, the image processing section 42 performs the predetermined image processing on the reflected light image RI outputted from the signal processing circuit 27 to produce a white light observation image and outputs the produced white light observation image to the display apparatus 5. Then, according to such operation of the image processing section 42, for example, a white light observation image having a hue that is substantially similar to a hue of an object such as living tissue when the object is viewed with a naked eye is displayed on the display apparatus 5.

On the other hand, the user administers the fluorescent dye FLA into the subject, for example, either before observation with FB light emitted from the fluorescent dye FLB or after completion of the observation with the FB light.

Subsequently, the user inserts the insertion portion 6 into the subject while viewing the white light observation image displayed on the display apparatus 5, and operates the input I/F 43 in a state in which the distal end portion of the insertion portion 6 is disposed in the vicinity of a desired site to be observed in the subject to provide an instruction to set the observation mode of the endoscope system 1A to the fluorescence observation mode.

Upon detection of the setting to the fluorescence observation mode, the control section 44 produces an illumination control signal for causing a simultaneous exit of WL light, EA light and EB light from the light source apparatus 3 and outputs the illumination control signal to the light source control section 34. Also, upon detection of the setting to the fluorescence observation mode, the control section 44 controls the image pickup device drive section 41 to drive the image pickup devices 24 and 25 in the camera unit 22A.

The light source control section 34 performs control to bring each of the white light source 31A, the excitation light source 31B and the excitation light source 31C into the on state, in response to the illumination control signal outputted from the control section 44. Also, the image pickup device drive section 41 produces image pickup device drive signals for causing image pickup operation and outputs the image pickup device drive signals to the image pickup device 24 and the image pickup device 25, respectively, according to control performed by the control section 44.

Then, as a result of the above-described operation being performed in the light source control section 34, WL light, EA light and EB light are radiated to the object, and WLR light, FA light, EAR light, which is reflected light emitted from the object in response to the radiation of the EA light, and EBR light, which is reflected light emitted from the object in response to the radiation of the EB light, enter from the objective lens 17 as return light, and the return light exiting through the objective lens 17 enters the relay lens 18.

Here, according to the above-described configuration of the optical viewing tube 21A, the optical filter 61 provided in the relay lens 18 has an optical characteristic such as indicated in FIG. 3 as an example, and thus, the WLR light in the wavelength band Wab and the FA light in the wavelength band Wcd each enter the optical splitter 23 through the eyepiece lens 19 side, while the EAR light in the wavelength band Wbc and the EBR light in the wavelength band Wde are blocked by the optical filter 61.

Therefore, when the observation mode of the endoscope system 1A is set to the fluorescence observation mode in a state in which the fluorescent dye FLA is administered into the subject, the WLR light exiting through the optical splitter 23 is subjected to image pickup by the image pickup device 24, and the FA light exiting through the optical splitter 23 is subjected to image pickup by the image pickup device 25, and the reflected light image RI, and a fluorescence image FAI, which is an image obtained by the image pickup of the FA light, are outputted to the image processing section 42, respectively, through the signal processing circuit 27.

According to control performed by the control section 44, the image processing section 42 performs the predetermined image processing on the reflected light image RI and the fluorescence image FAI outputted from the signal processing circuit 27 to produce a fluorescence observation image and outputs the produced fluorescence observation image to the display apparatus 5. Then, according to such operation of the image processing section 42, for example, the fluorescence observation image with information added to the white light observation image, the information indicating a state of generation of the FA light at the desired site to be observed in the subject, is displayed on the display apparatus 5.

Here, the image processing section 42 in the present embodiment is not limited to an image processing section configured to produce a fluorescence observation image using a reflected light image RI, and may be, for example, an image processing section configured to produce a fluorescence observation image without using a reflected light image RI, that is, using a fluorescence image FAI alone. Then, according to such operation of the image processing section 42, for example, a fluorescence observation image that enables a state of generation of FA light at a desired site to be observed in a subject to be directly viewed is displayed on the display apparatus 5.

On the other hand, the user administers a fluorescent dye FLB into the subject, for example, either before observation with the FA light emitted from the fluorescent dye FLA or after completion of the observation with the FA light.

Subsequently, the user inserts the insertion portion 6 into the subject while viewing the white light observation image displayed on the display apparatus 5, and operates the input I/F 43 in a state in which the distal end portion of the insertion portion 6 is disposed in the vicinity of the desired site to be observed in the subject to provide an instruction to set the observation mode of the endoscope system 1A to the fluorescence observation mode.

Upon detection of the setting to the fluorescence observation mode, the control section 44 produces an illumination control signal for causing a simultaneous exit of WL light, EA light and EB light from the light source apparatus 3 and outputs the illumination control signal to the light source control section 34. Also, upon detection of the setting to the fluorescence observation mode, the control section 44 controls the image pickup device drive section 41 to drive the image pickup devices 24 and 25 in the camera unit 22A.

The light source control section 34 performs control to bring each of the white light source 31A, the excitation light source 31B and the excitation light source 31C into the on state, in response to the illumination control signal outputted from the control section 44. Also, the image pickup device drive section 41 produces image pickup device drive signals for causing image pickup operation and outputs the image pickup device drive signals to the image pickup device 24 and the image pickup device 25, respectively, according to control performed by the control section 44.

Then, as a result of the above-described operation being performed in the light source control section 34, WL light, EA light and EB light are radiated to the object, and WLR light, FB light, EAR light and EBR light enter from the objective lens 17 as return light, and the return light exiting through the objective lens 17 enters the relay lens 18.

Here, according to the above-described configuration of the optical viewing tube 21A, the optical filter 61 provided in the relay lens 18 has an optical characteristic such as indicated in FIG. 3 as an example, and thus, the WLR light in the wavelength band Wab and the FB light in the wavelength band Wef each enter the optical splitter 23 through the eyepiece lens 19 side, while the EAR light in the wavelength band Wbc and the EBR light in the wavelength band Wde are blocked by the optical filter 61.

Therefore, when the observation mode of the endoscope system 1A is set to the fluorescence observation mode in a state in which the fluorescent dye FLB is administered into the subject, the WLR light exiting through the optical splitter 23 is subjected to image pickup by the image pickup device 24, and the FB light exiting through the optical splitter 23 is subjected to image pickup by the image pickup device 25, and the reflected light image RI, and a fluorescence image FBI, which is an image obtained by the image pickup of the FB light, are outputted to the image processing section 42, respectively, through the signal processing circuit 27.

According to control performed by the control section 44, the image processing section 42 performs the predetermined image processing on the reflected light image RI and the fluorescence image FBI outputted from the signal processing circuit 27 to produce a fluorescence observation image, and outputs the produced fluorescence observation image to the display apparatus 5. Then, according to such operation of the image processing section 42, for example, the fluorescence observation image with information added to the white light observation image, the information indicating a state of generation of the FB light at the desired site to be observed in the subject, is displayed on the display apparatus 5.

Also, the image processing section 42 according to the present embodiment is not limited to an image processing section configured to produce a fluorescence observation image using a reflected light image RI, and may be, for example, an image processing section configured to produce a fluorescence observation image without using a reflected light image R1, that is, using a fluorescence image FBI alone. Then, according to such operation of the image processing section 42, for example, a fluorescence observation image that enables a state of generation of FB light at a desired site to be observed in a subject to be directly viewed is displayed on the display apparatus 5.

As described above, according to the present embodiment, light travelling through the optical viewing tube 21A is filtered by the optical filter 61, and thus, for example, even though no filter for blocking EAR light and EBR light is provided on each of front faces of the image pickup devices 24 and 25, a white light observation using WLR light emitted from an object in response to radiation of WL light, a fluorescence observation using FA light emitted from a fluorescent dye FLA in response to radiation of EA light and a fluorescence observation using FB light emitted from a fluorescent dye FLB in response to radiation of EB light can be performed. Therefore, the present embodiment enables reduction in costs incurred in building a system that can be used for both white light observation and fluorescence observation responsive to plural types of fluorescent dyes.

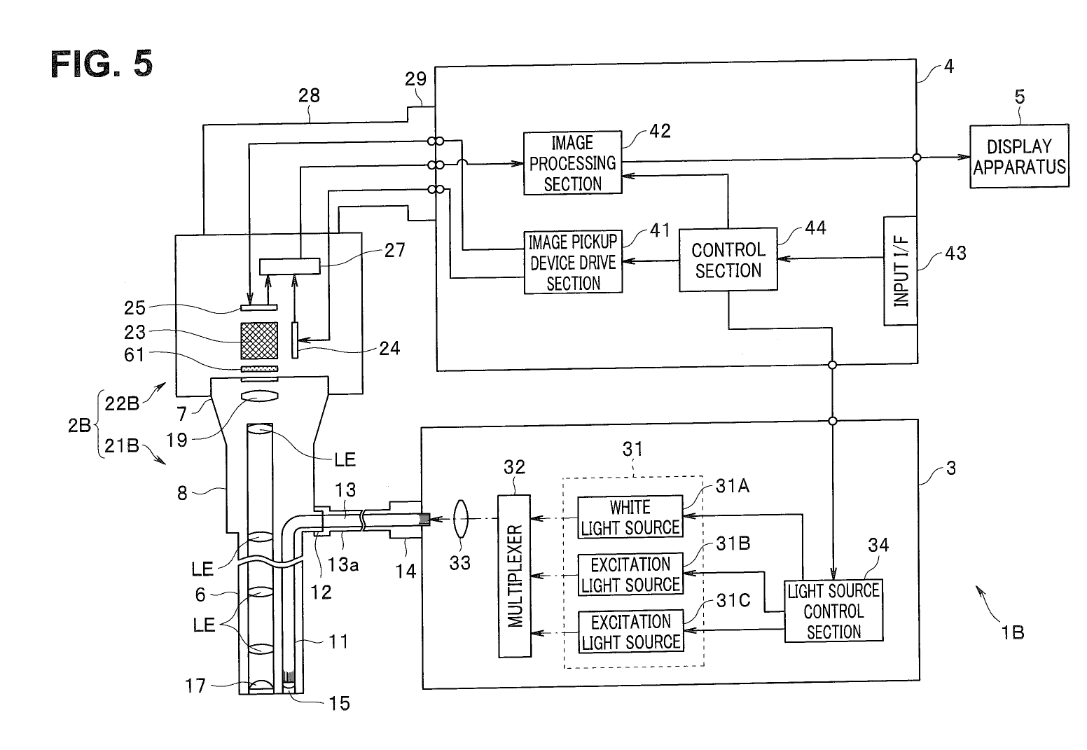

Here, the endoscope system 1A according to the present embodiment may be configured as, for example, an endoscope system 1B including an endoscope apparatus 2B instead of the endoscope apparatus 2A, which is illustrated in FIG. 5. A configuration of the endoscope system 1B according to a modification of the present embodiment will be described below. However, in the following, for simplicity, specific description of parts to which above-described components can be applied will be omitted where appropriate. FIG. 5 is a diagram illustrating a configuration of a major part of an endoscope system according to a modification of the first embodiment.

As illustrated in FIG. 5, the endoscope system 1B includes an endoscope apparatus 2B configured to be inserted into a subject and output an image obtained by image pickup of an object such as living tissue in the subject, a light source apparatus 3 configured to supply light to be radiated to the object, to the endoscope apparatus 2B, a video processor 4 configured to perform predetermined image processing on the image outputted from the endoscope apparatus 2B to produce an observation image or the like and output the observation image or the like, and a display apparatus 5.

The endoscope apparatus 2B is configured to enable observation using reflected light of white light, the reflected light being generated in response to radiation of the white light to an object present in a subject, and observation using fluorescence generated in response to radiation of excitation light to a fluorescent dye administered into the subject. Also, the endoscope apparatus 2B is configured to include an optical viewing tube 21B including an elongated insertion portion 6, and a camera unit 22B that is detachably attachable to an eyepiece portion 7 of the optical viewing tube 21B.

The optical viewing tube 21B has a configuration that is substantially similar to the configuration of the optical viewing tube 21A with the relay lens 18 from which the optical filter 61 is removed.

The camera unit 22B has a configuration that is substantially similar to the configuration of the camera unit 22A with an optical filter 61 added, the optical filter 61 having an optical characteristic such as indicated in FIG. 3. More specifically, the camera unit 22B is configured to include, for example, an optical filter 61 disposed at a predetermined position on the light entrance side of an optical splitter 23. In other words, the optical filter 61 in the present modification is disposed between an eyepiece lens 19 and the optical splitter 23 (or on a front face of the optical splitter 23) positioned on an optical path on which light entered from an objective optical system 17 travels to the optical splitter 23 through a relay lens 18.

Here, detailed description of operation of the respective sections of the endoscope system 1B will be omitted because the description is similar to the above-described content of the operation of the respective sections of the endoscope system 1A partially changed according to the position of disposition of the optical filter 61 in the endoscope apparatus 2B.

As described above, according to the present modification, light entering the optical splitter 23 in the camera unit 22B through the optical viewing tube 21B is filtered by the optical filter 61, and thus, for example, even if no filter for blocking EAR light and EBR light is provided on each of front faces of image pickup devices 24 and 25, a white light observation using WLR light emitted from an object in response to radiation of WL light, a fluorescence observation using FA light emitted from a fluorescent dye FLA in response to radiation of EA light, and a fluorescence observation using FB light emitted from a fluorescent dye FLB in response to radiation of EB light can be performed. Also, according to the present modification, the optical filter 61 is provided in the camera unit 22B, and thus, many of existing optical viewing tubes can be used as the optical viewing tube 21B in the endoscope system 1B. Therefore, the present modification enables reduction in costs incurred in building a system that can be used for both white light observation and fluorescence observation responsive to plural types of fluorescent dyes.

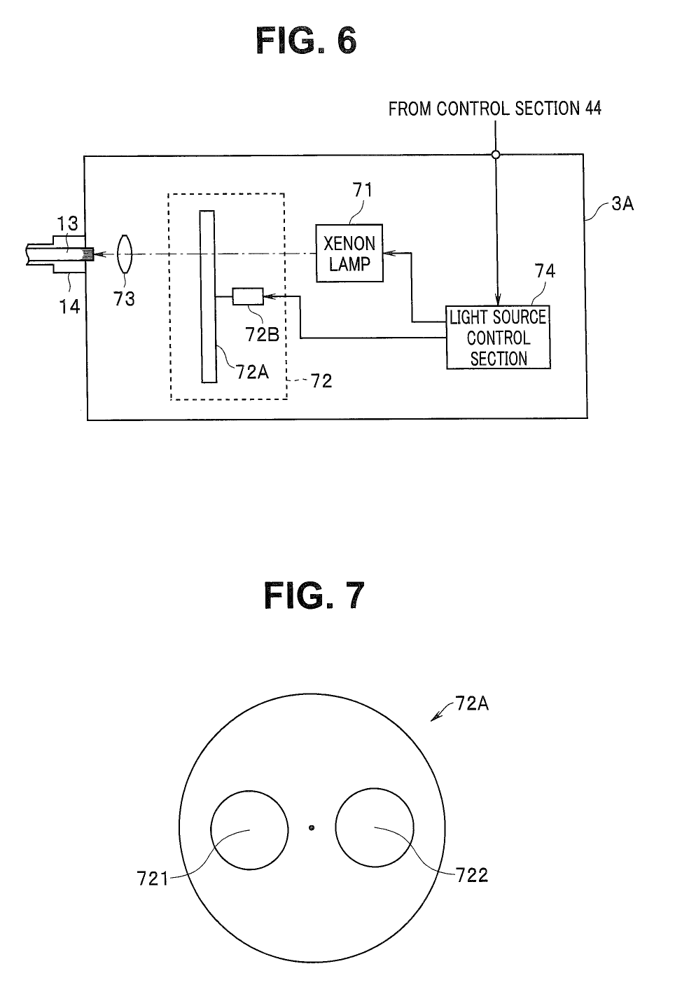

Also, according to the present embodiment, the endoscope system 1A or 1B may be configured using, for example, a light source apparatus 3A having the configuration illustrated in FIG. 6 instead of the light source apparatus 3. FIG. 6 is a diagram illustrating an example of the configuration of the light source apparatus according to the first embodiment, the example being different from the example in FIG. 2.

As illustrated in FIG. 6, the light source apparatus 3A is configured to include a xenon lamp 71, a filter switching device 72, a condenser lens 73 and a light source control section 74.

The xenon lamp 71 is configured to emit, for example, BL light, which is light in a broad band including a band of from the wavelength Wa to the wavelength We. Also, the xenon lamp 71 is configured to switch into an on state or an off state according to control performed by the light source control section 74. Also, the xenon lamp 71 is configured to generate an amount of BL light according to control performed by the light source control section 74 in the on state.

The filter switching device 72 is configured to include a rotary filter 72A provided so as to perpendicularly cross an optical path of light emitted from the xenon lamp 71, and a motor 72B configured to be driven to rotate according to control performed by the light source control section 74, to switch a filter inserted on the optical path of the light emitted from the xenon lamp 71 to one of filters in the rotary filter 72A.

The rotary filter 72A is formed to have, for example, a circular disc shape. Also, in the rotary filter 72A, for example, as illustrated in FIG. 7, a white light observation filter 721 and a fluorescence observation filter 722 are provided. FIG. 7 is a diagram illustrating an example of a configuration of a rotary filter provided in the light source apparatus in FIG. 6.

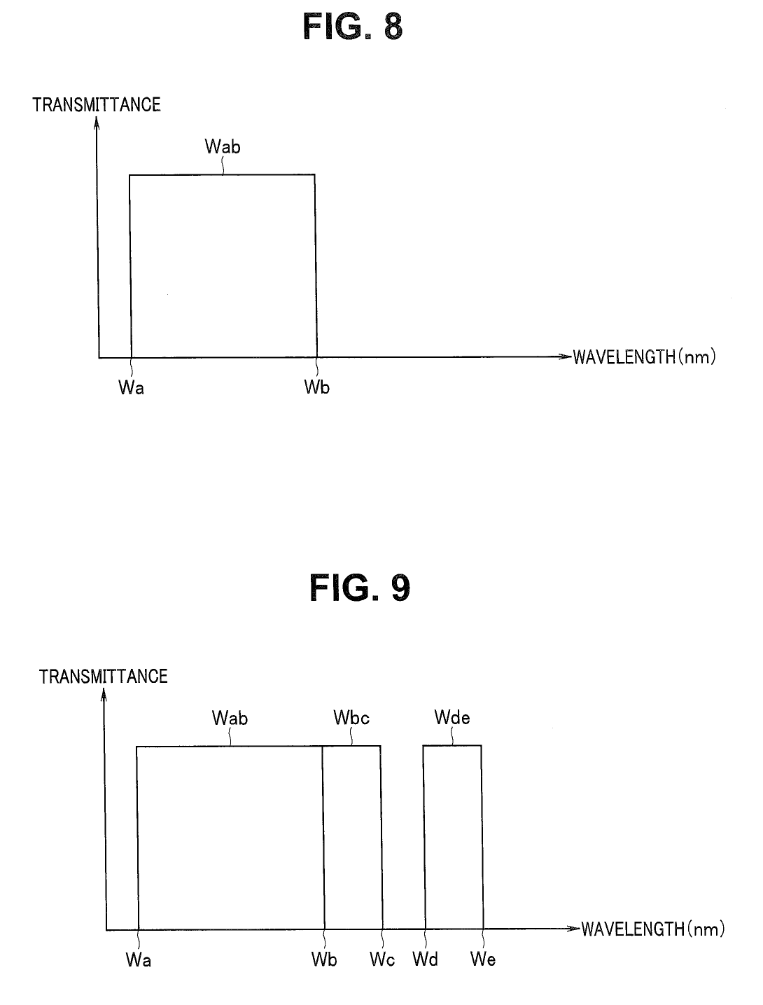

The white light observation filter 721 is formed to have an optical characteristic of, for example, as illustrated in FIG. 8, transmitting light included in a wavelength band Wab while blocking light included in a wavelength band other than the wavelength band Wab. FIG. 8 is a diagram indicating an example of a transmission characteristic of a white light observation filter provided in the rotary filter in FIG. 7.

The fluorescence observation filter 722 is formed to have an optical characteristic of, for example, as illustrated in FIG. 9, transmitting light included in any of three wavelength bands that are the wavelength band Wab, a wavelength band Wbc and a wavelength band Wde while blocking light included in a wavelength band other than the three wavelength bands. FIG. 9 is a diagram indicating an example of a transmission characteristic of a fluorescence observation filter provided in the rotary filter in FIG. 7.

In other words, the filter switching device 72 is configured to be capable of inserting one filter of the white light observation filter 721 and the fluorescence observation filter 722 on the optical path of the light emitted from the xenon lamp 71 while withdrawing the other filter that is different from the one filter from the optical path, by driving the motor 72B to rotate according to control performed by a light source control section 74.

The condenser lens 73 is configured to collect light entered through the filter switching device 72 and output the light to the light guide 13.

The light source control section 74 is configured to perform control of the xenon lamp 71 and the filter switching device 72 based on an illumination control signal outputted from the control section 44 in the video processor 4.

More specifically, the light source control section 74 is configured to, based on an illumination control signal outputted from the control section 44, for example, when it is detected that an observation mode of the endoscope system 1A or 1B is set to a white light observation mode, control the xenon lamp 71 to generate a predetermined amount of BL light and control the motor 72B in the filter switching device 72 to insert the white light observation filter 721 on the optical path of the light emitted from the xenon lamp 71. Also, the light source control section 74 is configured to, based on an illumination control signal outputted from the control section 44, for example, when it is detected that the observation mode of the endoscope system 1A or 1B is set to a fluorescence observation mode, control the xenon lamp 71 to generate a predetermined amount of BL light and control the motor 72B in the filter switching device 72 to insert the fluorescence observation filter 722 on the optical path of the light emitted from the xenon lamp 71.

Then, according to the present embodiment, even if the endoscope system 1A or 1B is configured using the light source apparatus 3A having the above-described configuration instead of the light source apparatus 3, effects that are similar to the above-described effects can be provided.

### Second Embodiment

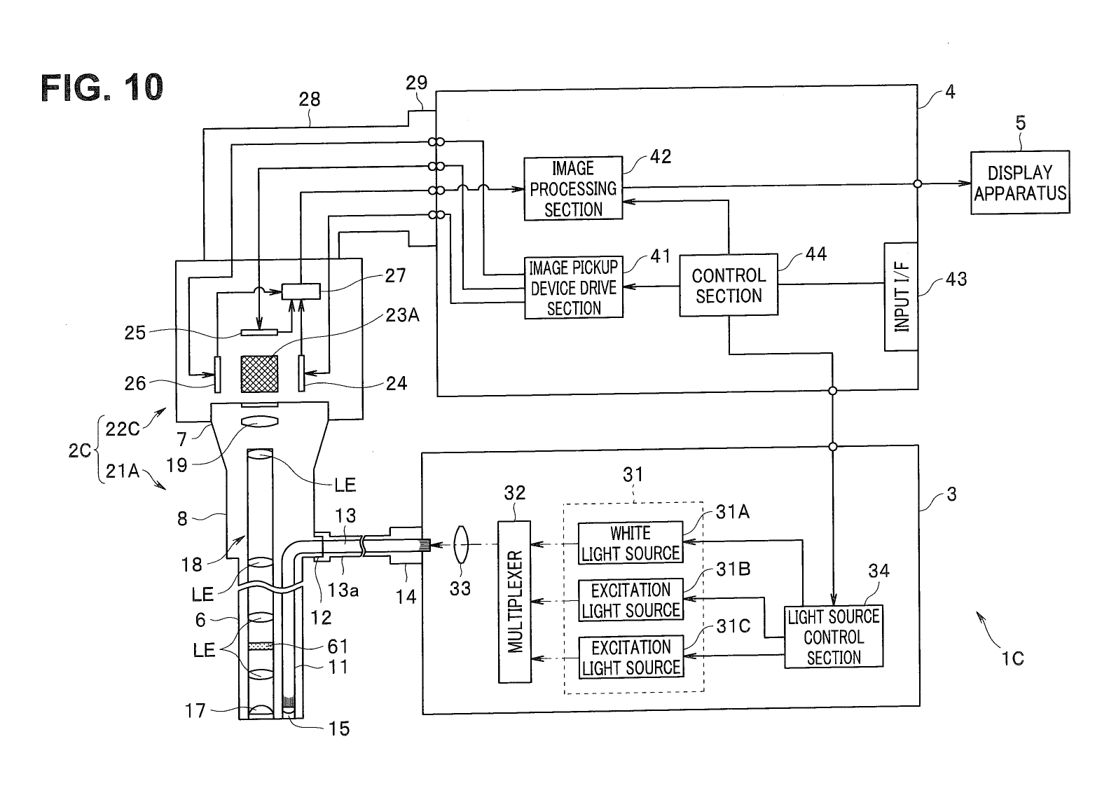

FIG. 10 relates to a second embodiment of the present invention. Note that, in the present embodiment, detailed description of parts having a configuration, etc., that are similar to the configuration, etc., in the first embodiment will be omitted, and description will be provided mainly on parts having a configuration, etc., that are different from the configuration, etc., in the first embodiment.

As illustrated in FIG. 10, an endoscope system 1C includes an endoscope apparatus 2C configured to be inserted into a subject and output an image obtained by image pickup of an object such as living tissue in the subject, a light source apparatus 3 configured to supply light to be radiated to the object, to the endoscope apparatus 2C, a video processor 4 configured to perform predetermined image processing on the image outputted from the endoscope apparatus 2C to generate an observation image or the like and output the observation image or the like, and a display apparatus 5. FIG. 10 is a diagram illustrating a configuration of a major part of an endoscope system according to a second embodiment.

The endoscope apparatus 2C is configured to enable observation using reflected light of white light, the reflected light being generated in response to radiation of the white light to an object present in a subject, and observation using fluorescence generated in response to radiation of excitation light to a fluorescent dye administered into the subject. Also, the endoscope apparatus 2C is configured to include an optical viewing tube 21A, and a camera unit 22C that is detachably attachable to an eyepiece portion 7 of the optical viewing tube 21A.

The camera unit 22C is configured to include an optical splitter 23A, image pickup devices 24, 25 and 26, and a signal processing circuit 27.

The optical splitter 23A is configured to include, for example, one or more optical members, for example, dichroic mirrors or prisms, and split light exiting through an eyepiece lens 19 into light in a plurality of mutually-different wavelength bands and cause respective exits of the light to the image pickup devices 24, 25 and 26. More specifically, the optical splitter 23A is configured to split light exiting through the eyepiece lens 19 into light in a first wavelength band, which is light in a band of wavelengths that are equal to or shorter than a wavelength Wb, light in a second wavelength band, which is light in a band of wavelengths that are longer than the wavelength Wb and equal to or shorter than a wavelength Wd, and light in a third wavelength band, which is light in a band of wavelengths that are longer than the wavelength Wd, and cause an exit of the first light to the image pickup device 24, cause an exit of the second light to the image pickup device 25 and causes an exit of the third light to the image pickup device 26.

The image pickup device 24 in the camera unit 22C is configured by, for example, a color CCD with a primary color or complimentary color filter provided on an image pickup surface. Also, the image pickup device 24 in the camera unit 22C is configured to perform image pickup of light exiting through the optical splitter 23A, the light being in a band of wavelengths that are equal to or shorter than the wavelength Wb, and produce and output an image according to the light subjected to the image pickup.

The image pickup device 25 in the camera unit 22C is configured by, for example, a highly-sensitive monochrome CCD. Also, the image pickup device 25 in the camera unit 22C is configured to perform image pickup of light exiting through the optical splitter 23A, the light being in a band of wavelengths that are longer than the wavelength Wb and equal to or shorter than the wavelength Wd, and produce and output an image according to the light subjected to the image pickup.

The image pickup device 26 in the camera unit 22C is configured by, for example, a highly-sensitive monochrome CCD. Also, the image pickup device 26 in the camera unit 22C is configured to perform image pickup operation according to an image pickup device drive signal outputted from the video processor 4. Also, the image pickup device 26 in the camera unit 22C is configured to perform image pickup of light exiting through the optical splitter 23A, the light being in a band of wavelengths that are longer than the wavelength Wd, and produce and output an image according to the light subjected to the image pickup.

The signal processing circuit 27 in the camera unit 22C is configured to perform predetermined signal processing such as, for example, correlated double sampling processing, gain adjustment processing and A/D conversion processing on the respective images outputted from the image pickup devices 24, 25 and 26. Also, the signal processing circuit 27 in the camera unit 22C is configured to output the image resulting from the aforementioned predetermined signal processing to the video processor 4 to which a signal cable 28 is connected.

Next, operation, etc., of the endoscope system 1C according to the present embodiment will be described. Here, the present embodiment will be described taking the case where a fluorescent dye FLA and a fluorescent dye FLB are administered substantially simultaneously into a subject, as an example.

First, a user connects the respective sections of the endoscope system 1C and turns on the power, and then operates an input I/F 43 to provide an instruction to set an observation mode of the endoscope system 1C to a white light observation mode.

Upon detection of the setting to the white light observation mode, a control section 44 produces an illumination control signal for causing an exit of WL light from the light source apparatus 3 and outputs the illumination control signal to a light source control section 34. Also, upon detection of the setting to the white light observation mode, the control section 44 controls an image pickup device drive section 41 to drive the image pickup device 24 in the camera unit 22C and stop driving of the image pickup devices 25 and 26 in the camera unit 22C.

In response to the illumination control signal outputted from the control section 44, the light source control section 34 performs control for bringing a white light source 31A into an on state and bringing each of an excitation light source 31B and an excitation light source 31C into an off state. Also, according to control performed by the control section 44, the image pickup device drive section 41 produces an image pickup device drive signal for causing image pickup operation and outputs the image pickup device drive signal to the image pickup device 24, and produces an image pickup device drive signal for stopping image pickup operation and outputs the image pickup device drive signal to the image pickup devices 25 and 26.

Then, as a result of the above-described operation being performed in the light source control section 34, WL light is radiated to an object, and WLR light emitted from the object enters from an objective lens 17 as return light, and the WLR light exiting through the objective lens 17 enters a relay lens 18. Also, when the observation mode of the endoscope system 1C is set to the white light observation mode, the WLR light exiting through the optical splitter 23A is subjected to image pickup by the image pickup device 24, and a reflected light image RI obtained by the image pickup of the WLR light is outputted to an image processing section 42 through the signal processing circuit 27.

According to control performed by the control section 44, the image processing section 42 performs predetermined image processing on the reflected light image RI outputted from the signal processing circuit 27 to produce a white light observation image and outputs the produced white light observation image to the display apparatus 5. Then, according to such operation of the image processing section 42, for example, a white light observation image having a hue that is substantially similar to a hue of an object such as living tissue when the object is viewed with a naked eye is displayed on the display apparatus 5.

On the other hand, the user administers the fluorescent dyes FLA and FLB into the subject, for example, at a predetermined timing before the observation mode of the endoscope system 1C is set to a fluorescence observation mode.

The user inserts an insertion portion 6 into the subject while viewing the white light observation image displayed on the display apparatus 5, and operates the input I/F 43 in a state in which a distal end portion of the insertion portion 6 disposed in the vicinity of a desired site to be observed in the subject to provide an instruction to set the observation mode of the endoscope system 1C to the fluorescence observation mode.

Upon detection of the setting to the fluorescence observation mode, the control section 44 produces an illumination control signal for causing a simultaneous exit of WL light, EA light and EB light from the light source apparatus 3 and outputs the illumination control signal to the light source control section 34. Also, upon detection of the setting to the fluorescence observation mode, the control section 44 controls the image pickup device drive section 41 to drive the image pickup devices 24, 25 and 26 in the camera unit 22C.

The light source control section 34 performs control to bring each of the white light source 31A, the excitation light source 31B and the excitation light source 31C into an on state, in response to the illumination control signal outputted from the control section 44. Also, the image pickup device drive section 41 produces image pickup device drive signals for causing image pickup operation and outputs the image pickup device drive signals to the image pickup devices 24, 25 and 26, respectively, according to control performed by the control section 44.

Then, as a result of the above-described operation being performed in the light source control section 34, WL light, EA light and EB light are radiated to the object, and WLR light, FA light, FB light, EAR light and EBR light enter from the objective lens 17 as return light, and the return light exiting through the objective lens 17 enters the relay lens 18.

Here, according to the above-described configuration of the optical viewing tube 21A, an optical filter 61 provided in the relay lens 18 has an optical characteristic such as indicated in FIG. 3 as an example, and thus, the WLR light in a wavelength band Wab, the FA light in a wavelength band Wcd and the FB light in a wavelength band Wef each enter the optical splitter 23A through the eyepiece lens 19 side, while the EAR light in a wavelength band Wbc and EBR light in a wavelength band Wde are blocked by the optical filter 61.

Therefore, when the observation mode of the endoscope system 1C is set to the fluorescence observation mode in a state in which the fluorescent dyes FLA and FLB are administered into the subject, the WLR light exiting through the optical splitter 23A is subjected to image pickup by the image pickup device 24, the FA light exiting through the optical splitter 23A is subjected to image pickup by the image pickup device 25, and the FB light exiting through the optical splitter 23A is subjected to image pickup by the image pickup device 26, and the reflected light image RI, a fluorescence image FAI and a fluorescence image FBI are outputted to the image processing section 42, respectively, through the signal processing circuit 27.

According to control performed by the control section 44, the image processing section 42 performs the predetermined image processing on the reflected light image RI, the fluorescence image FAI and the fluorescence image FBI outputted from the signal processing circuit 27 to produce a fluorescence observation image and outputs the produced fluorescence observation image to the display apparatus 5. Then, according to such operation of the image processing section 42, for example, the fluorescence observation image with information added to the white light observation image, the information enabling individually recognition of a state of generation of the FA light at the desired site to be observed in the subject and a state of generation of the FB light at the desired site to be observed, is displayed on the display apparatus 5.

Here, the image processing section 42 in the present embodiment is not limited to an image processing section configured to produce a fluorescence observation image using a reflected light image RI, a fluorescence image FAI and a fluorescence image FBI, and may be, for example, an image processing section configured to produce a fluorescence observation image without using a reflected light image RI, that is, using at least one of a fluorescence image FAI and a fluorescence image FBI. Then, according to such operation of the image processing section 42, for example, a fluorescence observation image that enables a state of generation of FA light and/or FB light at a desired site to be observed in a subject to be directly viewed is displayed on the display apparatus 5.

As described above, according to the present embodiment, light travelling through the optical viewing tube 21A is filtered by the optical filter 61, and thus, for example, even though no filter for blocking EAR light and EBR light is provided on each of front faces of the image pickup devices 24, 25 and 26, a white light observation using WLR light emitted from an object in response to radiation of WL light, a fluorescence observation using FA light emitted from a fluorescent dye FLA in response to radiation of EA light and a fluorescence observation using FB light emitted from a fluorescent dye FLB in response to radiation of EB light can be performed. Therefore, the present embodiment enables reduction in costs incurred in building a system that can be used for both white light observation and fluorescence observation responsive to plural types of fluorescent dyes.

Also, according to the present embodiment, for example, disposing respective image pickup surfaces of the image pickup devices 25 and 26 at respective positions corresponding to a focal length of an optical system including the objective lens 17, the relay lens 18, the eyepiece lens 19 and the optical splitter 23A, enables enhancement in image quality of a fluorescence image FAI and a fluorescence image FBI.

On the other hand, according to the present embodiment, the endoscope system 1C may be configured using the light source apparatus 3A having the configuration described above in the first embodiment instead of the light source apparatus 3. Then, according to the present embodiment, even where the endoscope system 1C is configured using the light source apparatus 3A instead of the light source apparatus 3, effects that are similar to the above-described effects can be provided.

Also, the configuration of the present embodiment may be modified as appropriate to configure, for example, an endoscope apparatus 2C including an insertion portion 6 formed in an elongated shape that enables insertion of the insertion portion 6 into a body cavity of a subject, and including an objective lens 17, an optical filter 61, an optical splitter 23A, image pickup devices 24 to 26D, and an image pickup device 26E provided in the distal end portion of the insertion portion 6. Then, in such case, the endoscope apparatus 2C can be configured without using a relay lens 18 and an eyepiece lens 19. Also, if the endoscope apparatus 2C is configured as stated above, the optical filter 61 may be disposed between the objective lens 17 and the optical splitter 23A (or on a front face of the optical splitter 23A).

The present invention is not limited to the respective embodiments and modification described above, and it should be understood that various changes and applications are possible without departing from the spirit of the invention.

What is claimed is:

1. An endoscope apparatus enabling observation using reflected light of white light, the reflected light being generated in response to radiation of the white light to an object present in a subject, observation using first fluorescence generated in response to radiation of first excitation light to a first fluorescent dye administered into the subject, and observation using second fluorescence generated in response to radiation of second excitation light to a second fluorescent dye administered into the subject, the endoscope apparatus comprising:
an insertion portion configured to be insertable into the subject;
an objective optical system provided in a distal end portion of the insertion portion, and configured to allow entrance of the reflected light of the white light, the first fluorescence, the second fluorescence, reflected light of the first excitation light, the reflected light being generated in response to the radiation of the first excitation light, and reflected light of the second excitation light, the reflected light being generated in response to the radiation of the second excitation light, as return light;
a transmitting optical system provided on a proximal end side relative to the objective optical system in the insertion portion, the transmitting optical system including a plurality of lenses, and being configured to transmit the return light obtained from the objective optical system;
a camera unit configured to be detachably attachable to the insertion portion, the camera unit including a plurality of image pickup devices configured to allow entrance of the return light travelling through the transmitting optical system;
an optical splitter provided in the camera unit and configured to split the return light exiting through the transmitting optical system into light in a wavelength band of the white light and light in a wavelength band that is different from the wavelength band of the white light and cause an exit of the light in the wavelength band of the white light and an exit of the light in the wavelength band that is different from the wavelength band of the white light;
an optical filter disposed between the plurality of lenses in the transmitting optical system or disposed at a predetermined position on the return light entrance side of the optical splitter in the camera unit, the optical filter being formed to have an optical characteristic of transmitting the reflected light of the white light, the first fluorescence and the second fluorescence and blocking the reflected light of the first excitation light and the reflected light of the second excitation light;
a first image pickup device for picking up an image of the reflected light of the white light exiting through the optical filter and the optical splitter, the first image pickup device being included in the plurality of image pickup devices; and
a second image pickup device for picking up an image of the first fluorescence and the second fluorescence exiting through the optical filter and the optical splitter, the second image pickup device being included in the plurality of image pickup devices.

2. The endoscope apparatus according to claim 1, wherein:
the optical splitter is configured to further split the light in the wavelength band that is different from the wavelength band of the white light into light in a wavelength band of the first fluorescence and light in a wavelength band of the second fluorescence and cause respective exits of the light in the wavelength band of the first fluorescence and the light in the wavelength band of the second fluorescence; and
the second image pickup device includes an image pickup device configured to receive the light in the wavelength band of the first fluorescence, and an image pickup device configured to receive the light in the wavelength band of the second fluorescence.

3. An endoscope system comprising:
a light source apparatus configured to be capable of supplying white light for illuminating an object present in a subject, first excitation light for exciting a first fluorescent dye administered into the subject to generate first fluorescence, and second excitation light for exciting a second fluorescent dye administered into the subject to generate second fluorescence;
an insertion portion configured to be insertable into the subject;
an objective optical system provided in a distal end portion of the insertion portion, and configured to allow entrance of reflected light of the white light generated in response to radiation of the white light to the object, the first fluorescence, the second fluorescence, reflected light of the first excitation light, the reflected light being generated in response to the radiation of the first excitation light, and reflected light of the second excitation light, the reflected light being generated in response to the radiation of the second excitation light, as return light;
a transmitting optical system provided on a proximal end side relative to the objective optical system in the insertion portion, the transmitting optical system including a plurality of lenses, and being configured to transmit the return light obtained from the objective optical system;
a camera unit configured to be detachably attachable to the insertion portion, the camera unit including a plurality of image pickup devices configured to allow entrance of the return light travelling through the transmitting optical system;
an optical splitter provided in the camera unit and configured to split the return light exiting through the transmitting optical system into light in a wavelength band of the white light and light in a wavelength band that is different from the wavelength band of the white light and cause an exit of the light in the wavelength band of the white light and an exit of the light in the wavelength band that is different from the wavelength band of the white light;
an optical filter disposed between the plurality of lenses in the transmitting optical system or disposed at a predetermined position on the return light entrance side of the optical splitter in the camera unit, the optical filter being formed to have an optical characteristic of transmitting the reflected light of the white light, the first fluorescence and the second fluorescence and blocking the reflected light of the first excitation light and the reflected light of the second excitation light;
a first image pickup device for picking up an image of the reflected light of the white light exiting through the optical filter and the optical splitter, the first image pickup device being included in the plurality of image pickup devices; and
a second image pickup device for picking up an image of the first fluorescence and the second fluorescence exiting through the optical filter and the optical splitter, the second image pickup device being included in the plurality of image pickup devices.
